(19) United States
(12) Patent Application Publication
(10) Pub. No.: US 2008/0182996 A1
(43) Pub. Date: Jul. 31, 2008

(54) Method For Separating Maleic Anhydride From Mixtures Containing the Same by Means of Stripping

(75) Inventors: Gunther Windecker, Ludwigshafen (DE); Alexander Weck, Freinsheim (DE); Michael Steiniger, Neustadt (DE); Jan Peter Bredehoft, Hassloch (DE); Bianca Bender, Worms (DE); Peter Knappik, Frankenthal (DE); Dany Vanden Hautte, Bernissart (BE); Bernard Pierre, Mont-Saint-Guibert (BE); Beatrix Haffner, Ludwigshafen (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/572,711

(22) PCT Filed: Jun. 30, 2006
(86) PCT No.: PCT/EP2006/063744

§ 371 Date: Oct. 29, 2007

(30) Foreign Application Priority Data
Jul. 5, 2005 (DE) 10 2005 031 314.0

(51) Int. Cl. C07D 307/33; C07C 27/06

(52) U.S. Cl. 549/262; 568/864

(57) ABSTRACT

The invention relates to a process for separating off maleic anhydride from a gas stream comprising maleic anhydride, in which
a) the gas stream comprising maleic anhydride is brought into contact with a liquid absorbent phase comprising at least one high-boiling inert absorption medium for maleic anhydride and
b) maleic anhydride is separated off from the resulting liquid absorbate phase by stripping with a hydrogen-comprising gas as stripping medium in a column,
   wherein the stripping medium is fed in at two points on the column which are at least one theoretical plate apart.

The invention relates to a process for separating off maleic anhydride from a gas stream comprising maleic anhydride, a process for preparing maleic esters and a process for preparing hydrogenation products of maleic acid derivatives, which comprise separating off maleic anhydride from a gas stream comprising maleic anhydride.

Maleic anhydride is prepared industrially by catalytic oxidation of hydrocarbons such as benzene, butenes or butane by means of air. The gaseous reaction mixture obtained comprises maleic anhydride together with, in particular, water, carbon monoxide and carbon dioxide.

The maleic anhydride which is obtainable from the reactor offgases from the oxidation of hydrocarbons or the maleic acid derivatives obtained from maleic anhydride by reaction with alcohols, e.g. maleic esters, are frequently subjected to a subsequent hydrogenation to form butanediol, tetrahydrofuran or γ-butyrolactone. WO 97/43234 describes a process for preparing γ-butyrolactone, butane-1,4-diol and tetrahydrofuran, in which maleic anhydride is scrubbed from the reactor offgases comprising maleic anhydride which are obtained from the oxidation of hydrocarbons by means of a high-boiling inert organic solvent as absorption medium and maleic anhydride is separated off from the resulting absorption product mixture by stripping with a hydrogen gas stream. The maleic anhydride which has been stripped out subsequently goes to a gas-phase hydrogenation for the preparation of butanediol, tetrahydrofuran and γ-butyrolactone. A disadvantage of this process is the increased formation of free maleic acid or fumaric acid in the absorption medium circuit. To achieve a high efficiency in the scrubbing out of the maleic anhydride by means of the absorption medium, it is necessary for the absorption medium to be as cool as possible. However, the solubility of high-boiling undesirable by-products which can be formed during the process is also reduced by the lower temperature and deposits are formed. In the presence of even small amounts of water as are frequently present in the reactor offgases from the oxidation of hydrocarbons and are also scrubbed out by the absorption medium, free maleic acid is formed as by-product from maleic anhydride and this is corrosive. In particular at high temperatures and long residence times, free maleic acid tends to isomerize into the only very sparingly soluble fumaric acid which forms deposits, particularly in plant components such as heat exchangers and columns, and makes shutdown of the entire plant necessary for cleaning purposes.

It is an object of the invention to provide a process for separating off maleic anhydride from the reactor offgases comprising maleic anhydride which are obtained from the oxidation of hydrocarbons which is suitable for the preparation of maleic acid derivatives or of hydrogenation products thereof and significantly reduces the abovementioned disadvantages.

The invention starts out from a process for separating off maleic anhydride from a gas stream comprising maleic anhydride, in which the gas stream comprising maleic anhydride is brought into contact with a liquid absorbent phase comprising at least one high-boiling inert absorption medium for maleic anhydride and maleic anhydride is separated off from the resulting liquid absorbate phase by stripping with a hydrogen-comprising gas in a further column. In the process of the invention, the stripping medium is fed in at two points on the column which are at least one theoretical plate apart.

A definition of a theoretical plate may be found in, for example, E.-U. Schlünder, F. Thurner, Destillation, Absorption, Extraktion, Thieme Verlag 1986, page 66 and pages 131-132.

For the purposes of the present invention, the absorbent phase is the liquid mixture of absorption media. The absorbate phase is the absorbent phase laden with the absorbed substance, in this case maleic anhydride or reaction products thereof.

The liquid absorbent or absorbate phase comprises at least one high-boiling inert absorption medium for maleic anhydride. The inert high-boiling absorption medium generally has a boiling point which is at least 30° C., preferably at least 50° C., particularly preferably at least 70° C., above the boiling point of maleic anhydride. Suitable absorption media are described, for example, in WO 97/43234. Examples are high-boiling esters of phthalic acid, terephthalic acid or maleic acid, e.g. dimethyl, diethyl or dibutyl phthalate, dimethyl terephthalate or dibutyl maleate, aromatic hydrocarbons such as dibenzylbenzene, esters of cycloaliphatic acids, e.g. dibutyl hexahydrophthalate, also polymethylbenzophenones, ethylene glycol ethers and polysiloxane ethers. Preferred absorption media are esters of aromatic or cycloaliphatic dicarboxylic acids, particularly preferably esters of phthalic acid or terephthalic acid. The high-boiling absorption medium is generally present in excess in the absorbate phase. The weight ratio of absorption medium to maleic anhydride is generally from 1:1 to 100:1, preferably from 2:1 to 100:1, particularly preferably from 4:1 to 100:1.

Maleic anhydride is separated off from the absorbate phase by stripping with a hydrogen-comprising gas as stripping medium. For the purposes of the present invention, the term stripping refers to the separation of the absorbed substance present in the absorbate phase by means of a desorption auxiliary (stripping medium), with the stripping medium becoming enriched in the absorbed substance. The absorbate phase is correspondingly depleted in the absorbed substance and is thus regenerated. Suitable hydrogen-comprising gases are hydrogen and mixtures of hydrogen with gases which are inert under the reaction conditions, for example nitrogen, or further components which can be formed during the subsequent hydrogenation. Preference is given to using the last-mentioned hydrogen mixture.

The molar ratio of hydrogen to maleic anhydride is generally from 1:1 to 500:1, preferably from 5:1 to 400:1, particularly preferably from 10:1 to 400:1.

The total amount of hydrogen in standard cubic meters (standard m³) is divided between the upper and lower points of introduction in a ratio of from 1:1 to 1000:1, preferably from 1:1 to 100:1, particularly preferably from 1:1 to 30:1. It can be advantageous to use hydrogen-comprising gas for the larger substream and pure hydrogen for the smaller substream.

Stripping is generally carried out in countercurrent. Here, the absorbate phase laden with maleic anhydride travels in the opposite direction to the gaseous stripping medium and intensive mass and heat transfer between the descending liquid absorbate phase and the ascending hydrogen phase takes place. The liquid absorbate phase is gradually depleted in maleic anhydride and the hydrogen phase correspondingly becomes enriched in maleic anhydride or reaction products thereof. Stripping is preferably carried out in countercurrent in a column, for example a column comprising random packing, ordered packing or bubble cap trays. Preference is given to introducing the hydrogen used as stripping medium into the column at two points in the lower or middle section of the column and the absorbate phase laden with maleic anhydride in the upper section of the column. The absorbate phase depleted in maleic anhydride is obtained at the bottom of the column, and the hydrogen used as stripping medium together with the maleic acid derivatives is obtained at the top of the column.

The stripping medium is, according to the invention, fed into the column at least two points of introduction, preferably at two points. Introduction through the liquid phase in the column or particularly preferably introduction into the gas space above the liquid phase in the column is preferably selected as geometrically lower point of introduction. In the case of introduction through the liquid phase in the column, the hydrogen used as stripping medium cools the liquid phase and in this way reduces the formation of high-boiling by-products, i.e. by-products having a boiling point higher than that of maleic anhydride, for example fumaric acid. The second point of introduction into the column for the remaining stripping medium, the preferred hydrogen mixture, is located at least one theoretical plate higher. The stripping medium can be preheated before it enters the column. The substream of the stripping medium at the geometrically lower point of introduction is preferably at a lower temperature than the substream of the stripping medium at the upper point of introduction.

Here, the temperature of the stripping medium at the upper point of introduction is from 100 to 250° C., preferably from 100° C. to 230° C., particularly preferably from 130 to 230° C. The temperature of the stripping medium at the lower point of introduction is generally from 25° C. to 250° C., preferably from 25° C. to 230° C., particularly preferably from 25° C. to 200° C.

As a result of the introduction according to the invention of the hydrogen-comprising stripping medium at two points of introduction, only part of the total stripping medium required is fed into the gas space above the liquid phase or through the liquid phase. At a predetermined equal flow rate, the diameter of the column can be made smaller in this region, which brings a considerable cost advantage. In addition, the content of liquid phase is reduced as a result and the residence time of the maleic anhydride or the free maleic acid in the liquid phase is thus shortened and the formation of high-boiling by-products such as fumaric acid is therefore reduced.

Furthermore, it has surprisingly been found that low-boiling by-products, i.e. by-products having a boiling point lower than that of maleic anhydride, e.g. methanol, ethanol, butanol and tetrahydrofuran can be removed from the absorption medium 5-15 times more effectively by means of the process of the invention than by means of hydrogen stripping using only one point of introduction for the hydrogen above the bottom. The inert absorption medium for maleic anhydride can therefore be recycled significantly more often, if appropriate without further purification.

For the purposes of the invention, maleic acid derivatives are maleic anhydride, maleic acid, maleic monoesters, maleic diesters, fumaric acid, fumaric monoesters and fumaric diesters, with the esters being based on aliphatic C₁-C₄-alcohols. Preference is given to using esters and diesters of methanol, ethanol, n-propanol, isopropanol, n-butanol, isobutanol, sec-butanol, n-pentanol and isopentanol, particularly preferably of methanol, ethanol and n-butanol.

Stripping in the countercurrent mode corresponds to a preferred embodiment of the invention. In a particularly preferred embodiment, further separation stages for holding back the high-boiling inert absorption medium are present between the feed point for the absorbate phase and the top of the column. The depleted absorbate phase is obtained at the bottom and this can be recirculated to renewed absorption. A high degree of depletion is not necessary, but is desirable. It is, based on the maleic anhydride content of the inflow at the top, generally from 80 to 100%, preferably from 95 to 100%.

Stripping is generally carried out at temperatures above 100° C. at the bottom. It is preferably from 100 to 250° C., particularly preferably from 130 to 250° C., in the stripping step. The pressure in the stripping step is generally from 10 mbar to 100 bar, preferably from 100 mbar to 60 bar, particularly preferably from 300 mbar to 20 bar. An advantage of the process of the invention is the small proportion of free maleic or fumaric acid in the overhead product. In general, the proportion of free acid in the overhead product, based on the total amount of maleic acid derivatives, is dependent on the amount of water present.

The invention further provides a process for preparing maleic esters, which comprises the steps:
a) contacting of a gas stream comprising reactor offgases comprising maleic anhydride which are obtained from the preparation of maleic anhydride by oxidation of hydrocarbons with a liquid absorbent phase comprising at least one inert high-boiling absorption medium having a boiling point which is at least 30° C. higher than that of maleic anhydride,
b) separation of maleic anhydride from the resulting liquid absorbate phase by stripping with a hydrogen-comprising gas as stripping medium,
c) esterification of the maleic anhydride which has been separated off with an aliphatic

C₁-C₅-alcohol.

As aliphatic C₁-C₅-alcohol for the esterification, preference is given to using methanol, ethanol, n-propanol, isopropanol, n-butanol, isobutanol, sec-butanol, n-pentanol and isopentanol, particularly preferably methanol, ethanol and n-butanol.

The mixture obtained in the separation step b) or the mixture obtained by further esterification according to step c) of the above-described process or maleic acid derivatives isolated from this mixture by distillation can subsequently be subjected to a hydrogenation. Depending on the reaction conditions, 1,4-butanediol, tetrahydrofuran and/or γ-butyrolactone are obtained preferentially.

The esterification of maleic anhydride, maleic acid, maleic monoesters or carboxylic acids in general is described in EP-A 0 255 399, EP-A 0 454 719, DE-A 5 543 673, DE-A 19 607 953 or in “Organikum”, VEB Deutscher Verlag der Wissenschaften, 15th Edition, Berlin 1977, pp. 498-502. Maleic diesters are preferably prepared by the process of the invention.

The present invention also provides a process for preparing at least one compound selected from the group consisting of 1,4-butanediol, γ-butyrolactone and tetrahydrofuran, which comprises the steps:
a) contacting of a gas stream comprising reactor offgases comprising maleic anhydride which are obtained from the preparation of maleic anhydride by oxidation of hydrocarbons with a liquid absorbent phase comprising at least one inert high-boiling absorption medium having a boiling point which is at least 30° C. higher than that of maleic anhydride,
b) separation of maleic anhydride by stripping with a hydrogen-comprising gas as stripping medium,
c) if appropriate, esterification of the maleic anhydride which has been separated off with an aliphatic C₁-C₅-alcohol,
d) if appropriate, separation of the alcohol from the mixture comprising maleic acid derivatives obtained in step c),
e) hydrogenation of the maleic acid derivatives obtained.

The maleic anhydride obtained in the separation step b) can be hydrogenated directly. However, it is also possible to subject the mixture obtained in the separation step b) to an esterification with an alcohol, using the above-mentioned aliphatic C₁-C₅-alcohols, preferably methanol, ethanol and n-butanol, as esterification alcohols.

The mixture obtained in the separation step b) or by esterification according to step c) can be subjected directly to the hydrogenation. Preference is given to separating off the alcohol from the mixture according to step d) prior to the hydrogenation.

The hydrogenation of the maleic acid derivatives obtained can be carried out in the liquid phase or in the gas phase. The hydrogenation can be carried out using homogeneously dissolved catalysts, suspended catalysts or fixed-bed catalysts. In general, the hydrogenation catalysts used comprise one or more of the following elements:

copper, palladium, platinum, ruthenium, rhenium, cobalt, manganese, nickel, molybdenum and chromium. Such hydrogenation catalysts and the hydrogenations carried out using them are described, for example, in WO 97/43234, EP-A 0 552 463, EP-A 0 724 908, DE-A 2 501 499, BE 851 227, U.S. Pat. No. 4,115,919, EP-A 0 147 219, EP-A 0 417 867, U.S. Pat. No. 5,115,086 and EP-A 0 382 050. In the gas-phase hydrogenation, the temperature is generally from 150 to 250° C. and the pressure is from 5 to 100 bar. In the hydrogenation in the liquid phase, the temperature is preferably from 100 to 300° C. and the pressure is from 60 to 300 bar.

The alcohol bound in the maleic esters is liberated in the hydrogenation and can be recovered and used again.

In the process of the invention, the formation of fumaric acid is largely prevented, so that problems caused by deposits in the absorption medium circuit are largely avoided.

The invention is illustrated by the following examples.

### EXAMPLES

The experiments described below are carried out in an apparatus comprising a bottom vessel, packed column and condenser at the top whose diameter is 58 mm (high-pressure standard NW 58). The total height of the apparatus from the bottom vessel to the condenser at the top, inclusive, is about 6.7 m. The column is equipped with B1-500 packing elements from Montz and has 18 theoretical plates.

The absorption medium can, as a matter of choice, be fed in at three different points in the upper, middle and lower parts of the apparatus, but always so that packing elements are present both above and below the inlet.

The hydrogen-comprising stripping medium can be fed in at a plurality of points in the lower region of the column which are all located below the lowest feed point for the absorption medium.

The temperature-controlled condenser at the top makes it possible to condense substreams and return them to the column as runback.

The acid content was determined by means of the silylation method. For this purpose, a 0.05 g sample was admixed with 0.05 g of an internal standard (tetraethylene glycol dimethyl ether [TEGDME]) and 0.5 g of n,o-bis(trimethylsilyl)trifluoroacetamide (BSTFA) and the solution was heated at 80° C. for 25 minutes. The sample which had been treated in this way was analyzed by gas chromatography using an HP 6890 gas chromatograph (column DB-1, 60 m length). The water content was determined by the Karl Fischer method.

### Example 1

Before the beginning of the trial, the above-described apparatus was flushed with the absorption medium dibutyl phthalate, emptied of the residue and filled with fresh absorption medium (=0% of fumaric acid).

The circulating stream comprised 13 kg/h of dibutyl phthalate as absorption medium containing about 10% by weight of maleic anhydride and 1400 ppm of water and was fed into the middle section of the column. The hydrogen-comprising stripping medium (pure hydrogen or hydrogen gas mixture comprising >95% by volume of H₂) was fed in countercurrent into the lower part of the column, as follows:

The larger substream of the stripping medium (hydrogen gas mixture) was introduced at 27 standard m³/h and about 225° C. into the gas space above the liquid phase in the column.

The smaller substream of the stripping medium (pure hydrogen) was introduced at 1.5 standard m³/h and about 30° C. directly into the liquid phase in the column.

A temperature of 136° C. was established at the bottom, since the introduction of the smaller substream through the liquid phase produces an advantageous cooling action. After 72 hours, a stable end value of 0.22% by weight of fumaric acid in the bottom product was achieved. The water value was ˜130 ppm, and the residual maleic anhydride content was ˜0.45% by weight. At 136° C., the solid-liquid saturation of fumaric acid in dibutyl phthalate is ˜0.38% by weight.

### Comparative Example 2

Before the beginning of the trial, the above-described apparatus was flushed, emptied of the residue and filled with fresh absorption medium (=0% by weight of fumaric acid). The circulating stream comprised 13 kg/h of dibutyl phthalate as absorption medium containing about 10% by weight of maleic anhydride and 1400 ppm of water and was fed into the middle section of the column.

However, in contrast to example 1, the hydrogen-comprising stripping medium (hydrogen mixture comprising >95% by volume of H₂) was fed in countercurrent into the lower part of the column at only one point:

The entire stripping medium stream of 28.5 standard m³/h and about 225° C. was introduced into the gas space above the liquid phase in the column.

A temperature of 148° C. was established at the bottom. After 72 hours, a stable end value of 0.28% by weight of fumaric acid in the bottom product was achieved.

At 148° C., the solid-liquid saturation of fumaric acid in dibutyl phthalate is ˜0.48% by weight.

### Example 3

To examine the effects of residence time and temperature at the bottom on the formation of fumaric acid, batch experiments were carried out in an oil-heated 500 ml double-wall glass flask provided with a superposed reflux condenser (standard ground glass joint 29 mm, A=0.05 m²).

5% by weight of maleic acid in the absorption medium dibutyl phthalate were placed in the flask, the mixture was boiled under reflux and samples were analyzed at defined time intervals.

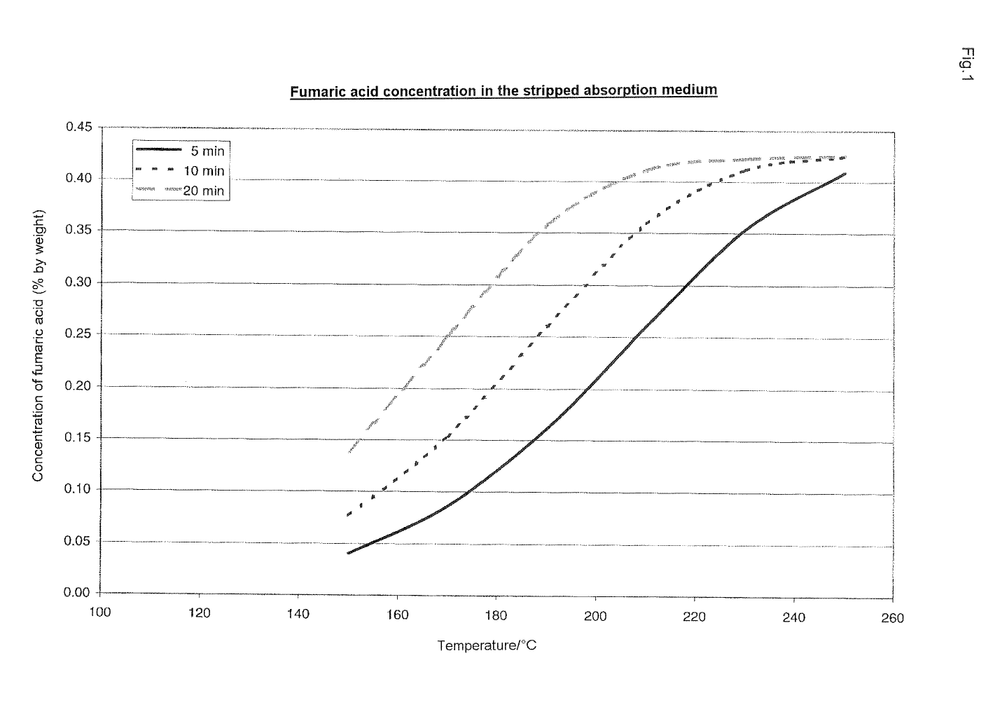

As can clearly be seen in FIG. 1, both the residence time in the liquid phase in the column and its temperature have a significant influence on the amount of fumaric acid formed.

1: A process for separating off maleic anhydride from a gas stream comprising maleic anhydride, in which
a) the gas stream comprising maleic anhydride is brought into contact with a liquid absorbent phase comprising at least one high-boiling inert absorption medium for maleic anhydride and
b) maleic anhydride is separated off from the resulting liquid absorbate phase by stripping with a hydrogen-comprising gas as stripping medium in a column,
wherein the stripping medium is fed in at two points on the column which are at least one theoretical plate apart.

2: The process according to claim 1, wherein part of the stripping medium is fed in through the liquid phase in the column or into the gas space above the liquid phase.

3: The process according to either claim 1, wherein stripping with a hydrogen-comprising gas is carried out at from 100 to 250° C. and a pressure of from 10 mbar to 100 bar.

4: The process according to claim 1, wherein reactor offgases comprising maleic anhydride which are obtained from the preparation of maleic anhydride by oxidation of hydrocarbons are used as gas stream.

5: The process according to claim 4 for preparing esters of maleic acid, which comprises the additional step of esterification of the maleic anhydride which has been separated off with an aliphatic C₁-C₅-alcohol.

6: The process according to claim 5 for preparing diesters of maleic acid.

7: The process according to claim 4 for preparing at least one compound selected from the group consisting of 1,4-butanediol, γ-butyrolactone and tetrahydrofuran, which comprises the additional steps
c) of optional, esterification of the maleic anhydride with an aliphatic C₁-C₅-alcohol,
d) of optional, separation of the alcohol from the resulting mixture comprising derivatives of maleic acid,
e) hydrogenation of the maleic acid derivatives obtained.
